(19) United States
(12) Patent Application Publication
(10) Pub. No.: US 2007/0239483 A1
(43) Pub. Date: Oct. 11, 2007

(54) Methods for individualized health assessment service

(75) Inventors: Mark B. Chandler, Austin, TX (US); George Rodgers, Austin, TX (US)

Correspondence Address: FOLEY AND LARDNER LLP;SUITE 500, 3000 K STREET NW, WASHINGTON, DC, 20007, US

(73) Assignee: BioPhysical Corporation

(21) Appl. No.: 11/651,954

(22) Filed: Jan. 9, 2007

Related U.S. Application Data
(60) Provisional application No. 60/757,039, filed on Jan. 9, 2006.

(51) Int. Cl. G06Q 10/00; C12M 1/34; A61B 5/00

(52) U.S. Cl. 705/2; 435/287.1; 600/300

(57) ABSTRACT

Provided is a Health Assessment Service comprising a marketing function, a testing function, an evaluation function, and a reporting function and a method of providing the Health Assessment Service. Also provided is a method of diagnosing a condition comprising measuring levels of biomarkers for the condition at a first and second time and comparing the change in levels of biomarkers to reference change values for the condition.

### CROSS-REFERENCE TO RELATED PATENT APPLICATIONS

This patent application claims the benefit under 35 U.S.C. § 119(e) of U.S. Provisional Application No. 60/757,039, filed Jan. 9, 2006, which is herein incorporated by reference in its entirety.

### FIELD OF INVENTION

This invention generally relates to methods for providing individualized health assessment service.

### BACKGROUND OF THE INVENTION

Individual patients, often suffering from or at a recognized risk for various conditions, routinely visit physicians for diagnosis and treatment. Such visits happen at outpatient clinics or hospitals and can be for preventive care, for monitoring of conditions for which the patient is known to be at risk, or for diagnosis and treatment of certain symptoms. Doctors commonly order laboratory tests such as lipid panels to screen for heart disease or blood glucose to screen for diabetes on patients in various age groups or other categories. Save for these types of narrowly targeted tests that are requested by a physician based upon a particular factor, individuals do not currently have direct access to a comprehensive assessment that informs them about the status of numerous biomarkers in their system or the potential correlation of such biomarkers toward predisposition to disease.

The options for individuals interested in ascertaining and understanding the levels of many biomarkers in their systems, including being alerted to biomarkers whose concentrations fall outside a normal range, are currently limited. Therefore, there is a need to provide directly to individuals an individualized health assessment service that furnishes understandable results to a broad test of numerous biomarkers that have pathological, therapeutic, or diagnostic relevance.

### SUMMARY OF THE INVENTION

The present invention provides a method for providing a Health Assessment Service (HAS). The method can be practiced by marketing the HAS to a consumer, initiating the HAS with the consumer, obtaining information and a biosample from the consumer, subjecting the biosample to one or more biomarker test panels, evaluating the results of the test panel or panels, generating a report for the customer containing the results and evaluation, and/or consulting with the consumer regarding the results and evaluation.

The present HAS invention is advantageous in one respect in that it allows individuals direct and convenient access to a comprehensive assessment of their total health picture, which is heretofore prohibitively impractical, time consuming, costly, or altogether unavailable.

The present invention has as another advantage in that it screens for numerous biomarkers that may indicate the presence of many medical conditions and diseases, such as cancer, cardiovascular disease, metabolic disorders, autoimmune disease, viral and bacterial diseases, and hormonal imbalance. In addition to biomarkers whose affects on health are broadly recognized, the HAS tests biomarkers whose effects or implications in health are recognized by clinical specialists and medical and scientific researchers. The present invention has as another advantage that it allows an individual consumer of the HAS and/or the individual's personal physician to monitor changes and trends in blood chemistry over time.

The present invention has as yet another advantage that a medical team reviews the results of the biomarker tests and provides a consultation with the individual consumer and/or the individual consumer's personal physician regarding the meaning and implications of the test results.

The present invention has still another advantage in that an understandable report is generated for the consumer, which comprises color codes and flagged biomarkers that are found to be present in levels different than those found in the normal population and therefore possibly indicative of a medical condition, a possible onset of a medical condition, or a predisposition to a medical condition that the consumer will want to seek additional diagnosis of, treatment for, or closer monitoring.

In a further embodiment, there is provided a Health Assessment Service (HAS) comprising:

(i) a marketing function that brings to the attention of potential consumers an ability to purchase an individualized health assessment service;

(ii) a testing function that obtains one or more test samples taken from a consumer who has elected to purchase the service, which one or more samples are subjected to one or more test panels, each of said one or more test panels comprising qualitative and/or quantitative tests for the presence or absence of a plurality of biomarkers in said one or more samples;

(iii) an evaluation function that reviews results from said tests and optionally generates one or more reports; and

(iv) a reporting function that communicates one or more reports to the consumer in a manner that brings to the consumer's attention test results that might inform of disease, medical condition, potential health risks and/or problems, if any.

In a further embodiment, there is provided a method of providing a Health Assessment Service (HAS) comprising:

(i) soliciting one or more consumers who might be interested in purchasing an individualized health assessment service;

(ii) obtaining one or more test samples taken from a consumer who has elected to purchase the service;

(iii) subjecting the one or more samples to one or more test panels, each of said one or more test panels comprising qualitative and/or quantitative tests for the presence or absence of a plurality of biomarkers in said one or more samples;

(iv) reviewing results from said tests and optionally generating one or more reports; and

(v) communicating one or more reports to the consumer in a manner that brings to the consumer's attention test results that might inform of disease, medical condition, potential health risks and/or problems, if any.

In a further embodiment, there is provided a method of diagnosing a condition selected from the group consisting of autoimmune disorder, cancer, cardiovascular disease, disease and repair associated with cell signaling, diabetes, endocrine condition, hematological abnormality, hormonal imbalance, immune reaction/inflammation, infectious disease, metabolic disorder, malnutrition, impaired organ function, and osteoarthritis in a patient comprising

(a) measuring levels of biomarkers in a test panel comprising one or more of biomarkers in a first sample from a patient at a first time,
wherein the biomarkers are associated with the condition;

(b) measuring levels of biomarkers in a second sample from the patient at a second time, wherein the biomarkers in (b) are the same as the biomarkers in (a);

(c) calculating a percentage change between the levels of biomarkers in (a) and (b); and

(d) comparing the percentage change in (c) to a reference change value; wherein
(i) percentage change in (c) that is equal to or higher than the reference change value indicates a possibility of a presence of the condition in the patient; or
(ii) percentage change in (c) that is lower than the reference change value indicates a decreased possibility of a presence of the condition in the patient.

### BRIEF DESCRIPTION OF THE DRAWINGS

FIG. 1 is a block diagram depicting the steps involved in the method of providing the Individualized Health Assessment Service. The method begins with acquiring customers via marketing, promoting, word of mouth, and branding. The method continues with the customer initiating the purchase of the service and providing to the provider of the service information necessary to create a customer profile. Next the customer submits to the provider necessary documents, including a Medical History Questionnaire and an Informed Consent Form. The customer then arranges a time and place to provide a biosample. For instance, the customer's blood sample is obtained and sent to a laboratory, preferably, the provider's laboratory, for testing. Results of the biomarker testing are analyzed by a medical team and a hard copy report is generated and sent to the customer. The customer then consults with the provider's medical team regarding the test results and implications.

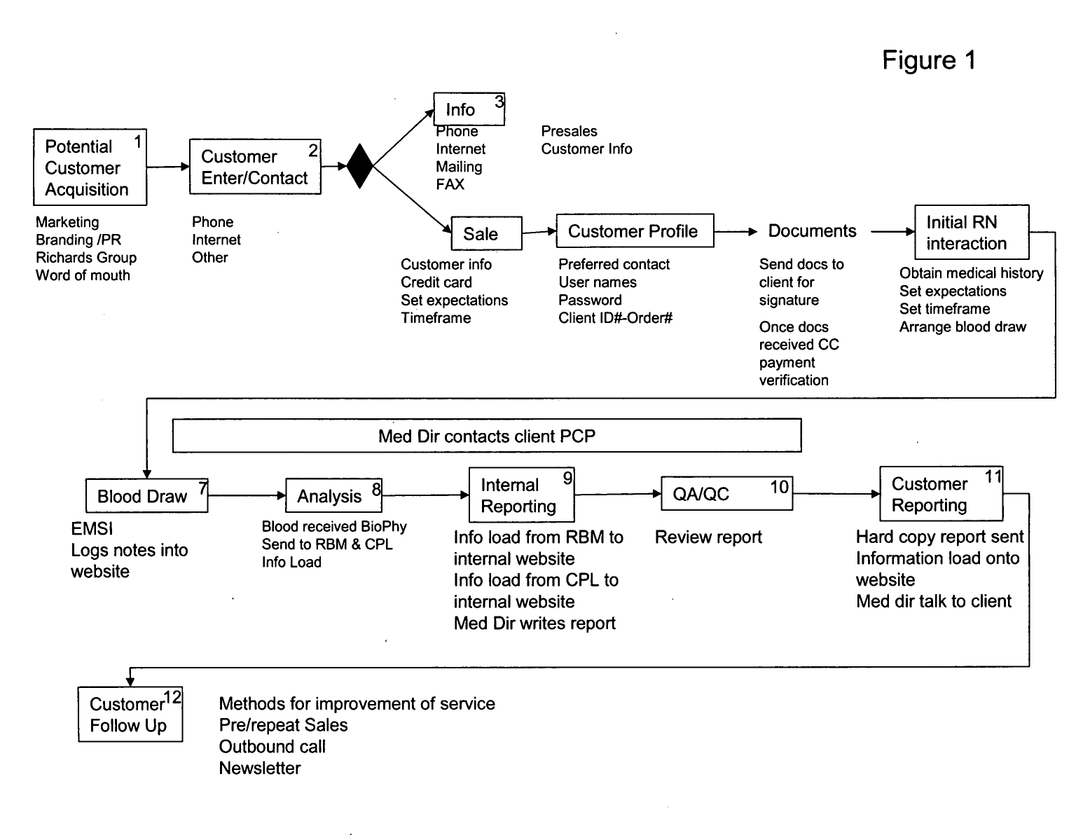

### DETAILED DESCRIPTION OF THE PREFERRED EMBODIMENTS

Provided herein is a method for providing a service that measures in a patient's blood or other sample a panel of analytes or biomarkers relevant to the pathology, treatment, risk, or diagnosis of various medical conditions. Generally, the present invention involves marketing the service to consumers, typically outwardly healthy or asymptomatic consumers, testing a panel of biomarkers on a sample of the consumer's blood, evaluating the test results, and reporting the results and evaluation to the consumer.

In a preferred embodiment, the marketing function comprises reaching potential consumers directly via internet or other advertising means that do not typically involve communicating through consumers' physicians, although word of mouth referrals are not precluded. Consumers who might be interested in purchasing an individualized health assessment service (“HAS”) are so solicited. These consumers may believe they are healthy and at no risk for any medical conditions, or they may know of one or more medical conditions affecting them or a family member or for which they are at risk. These consumers typically are interested in obtaining more information and data regarding their individual health, including the level at which each of a large number of biomarkers is present in their blood or other sample, whether the level for each specific biomarker falls within, above, or below a normal range observed in the population, how far outside the normal range any deviating biomarker lies, and what is medically known about the implications of any such deviating biomarker. In addition, for markers that lie within, above, or below a normal range, these consumers typically are interested in an understandable presentation of the latest knowledge that general practitioners or internists, specialists, and researchers possess about these biomarkers.

The testing function comprises obtaining one or more biological (e.g., whole blood, plasma, serum or urine and the like) samples taken from a consumer who has elected to purchase the HAS. The blood or other sample may be collected at a location specified by the consumer, such as at a health clinic or at the consumer's residence. The blood sample is carefully collected by a phlebotomist, nurse, or other trained health professional affiliated with, designated by, or approved by the provider of the HAS (“HAS Provider”). The blood sample is then carefully packed and shipped to the laboratory of the HAS Provider for testing and analysis. In the context of the present disclosure, “blood” includes any blood fraction, for example serum, that can be analyzed according to the methods described herein. Serum is a standard blood fraction that can be tested, and is tested in the Examples below. By measuring blood levels of a particular biomarker, it is meant that any appropriate blood fraction can be tested to determine blood levels and that data can be reported as a value present in that fraction. As a non-limiting example, the blood levels of a biomarker can be presented as 50 pg/mL serum.

In a preferred embodiment, the testing function further comprises the consumer completing a confidential Medical History Questionnaire (“Questionnaire”). The Questionnaire supplies the HAS Provider with important information about the consumer's current state of health, medical history, and family medical history. The Questionnaire and biological sample may be submitted to the HAS Provider simultaneously or independently.

In a preferred embodiment, the testing function further comprises the consumer completing an Informed Consent Form. The Informed Consent Form apprises the consumer of his or her rights regarding the confidentiality of personal and medical data. The Informed Consent Form also apprises the consumer of the procedures involved in the HAS, what are the limitations of the HAS, what are the risks of the HAS, and what are the alternatives to the HAS. The Informed Consent Form is to be completed preferably prior to the time the consumer's biological sample is scheduled to be collected.

Biomarkers

Identified below in Table 1 are certain sample fluid (e.g., blood) analytes or biomarkers useful to include as part of a broad assessment of one's personal health status. The majority of the following biomarkers are known to exist within a particular range of concentrations or levels in individuals from a basically healthy, asymptomatic, middle-aged population (“normal” individuals). Each of those biomarkers is further known to be relevant to the biochemistry, pathology, diagnosis, or treatment of, or the risk for, one or more medical conditions if the biomarker is present at a concentration significantly outside the range at which it is present in normal individuals. The concentration ranges presented represent merely exemplary examples of what may constitute normal ranges for these biomarkers and should not be construed as limiting what can be designated as normal ranges in the present or other assays. The remaining of the following biomarkers are normally absent from an individual's biosample. The presence of a detectable concentration of each of those biomarkers is known to be indicative of exposure to and contraction of certain infectious agents, for example viruses, and the possible presence of certain associated medical conditions.

TABLE 1
Analytes Included in Health Assessment Service

| | | Normal | |
|---|---|---|---|
| No. | Analyte | Range | Units |
| 1 | Glucose | 65-100 | mg/dL |
| 2 | Blood Urea Nitrogen | 8-25 | mg/dL |
| 3 | Creatinine | 0.8-1.4 | mgldL |
| 4 | Blood Urea Nitrogen/Creatinine Ratio | 6-28 | ratio |
| 5 | Uric Acid | 3.3-8.5 | mg/dL |
| 6 | Sodium | 133-146 | meq/L |
| 7 | Potassium | 3.5-5.3 | meq/L |
| 8 | Chloride | 97-110 | meq/L |
| 9 | Carbon Dioxide | 18-30 | meq/L |
| 10 | Calcium | 8.5-10.5 | mg/dL |
| 11 | Phosphorus | 2.2-4.5 | mg/dL |
| 12 | Magnesium | 1.3-2.3 | mg/dL |
| 13 | Cholesterol | <200 | mg/dL |
| 14 | Triglycerides | <150 | mg/dL |
| 15 | High Density Lipoprotein Cholesterol | >39 | mg/dL |
| 16 | Low Density Lipoprotein Cholesterol | <100 | mg/dL |
| 17 | Low Density Lipoprotein/High Density | <3.55 | ratio |
| | Lipoprotein Ratio | | |
| 18 | Total Protein | 6.0-8.4 | g/dL |
| 19 | Albumin | 2.9-5.0 | g/dL |
| 20 | Globulin | 2.0-3.8 | g/dL |
| 21 | Albumin/Globulin Ratio | 0.9-2.5 | ratio |
| 22 | Bilirubin, Total | 0.1-1.3 | mg/dL |
| 23 | Alkaline Phosphatase | 30-132 | U/L |
| 24 | Gamma Glutamyl Transferase | 7-70 | U/L |
| 25 | Aspartate Aminotransferase | 5-35 | U/L |
| 26 | Alanine Aminotransferase | 7-56 | U/L |
| 27 | Lactate Dehydrogenase | 60-225 | U/L |
| 28 | Creatine Kinase, Total | 37-289 | U/L |
| 29 | Amylase | 30-120 | U/L |
| 30 | Iron, Serum | 35-158 | ug/dL |
| 31 | Iron Binding Capacity, Unsaturated | 155-300 | ug/dL |
| 32 | Iron Binding Capacity, Total | 250-450 | ug/dL |
| 33 | Iron, Percent Saturated | 20-50 | |
| 34 | White Blood Cell Count | 4.0-11.0 | K/cumm |
| 35 | Red Blood Cell Count | 4.10-5.70 | M/cumm |
| 36 | Hemoglobin | 13.0-17.0 | g/dL |
| 37 | Hematocrit | 37.0-49.0 | % |
| 38 | Mean Corpuscular Volume | 80-100 | fL |
| 39 | Mean Corpuscular Hemoglobin | 27.0-34.0 | Uug |
| 40 | Mean Corpuscular Hemoglobin Concentration | 32.0-36.4 | g/dL |
| 41 | Red Cell Distribution Width | 11.0-15.0 | % |
| 42 | Neutrophil Count | 1.8-7 | K/cumm |
| 43 | Neutrophil Percentage | 40-74 | % |
| 44 | Lymphocyte Count | 1-4.8 | K/cumm |
| 45 | Lymphocyte Percentage | 19-48 | % |
| 46 | Monocyte Count | 0-0.8 | K/cumm |
| 47 | Monocyte Percentage | 3-11 | % |
| 48 | Eosinophil Count | 0-0.5 | K/cumm |
| 49 | Eosinophil Percentage | 0-7 | % |
| 50 | Basophil Count | 0-0.2 | K/cumm |
| 51 | Basophil Percentage | 0-2 | % |
| 52 | Platelet Count | 130-400 | K/cumm |
| 53 | Hemoglobin A1c | 4.0-6.0 | % |
| 54 | Hepatitis A Antibody | NEGATIVE | |
| 55 | Hepatitis B Core Antibody | NEGATIVE | |
| 56 | Hepatitis B Surface Antigen | NEGATIVE | |
| 57 | Hepatitis B Surface Antibody | NEGATIVE | |
| 58 | Hepatitis C Antibody | NEGATIVE | |
| 59 | Alpha Fetoprotein | 0.6-33 | ng/mL |
| 60 | Calcitonin | <23 | pg/mL |
| 61 | Cancer Antigen 125 | <35 | U/mL |
| 62 | Cancer Antigen 15-3 (BR-MA) | <53 | U/mL |
| 63 | Cancer Antigen 19-9 | <37 | U/mL |
| 64 | Carcinoembryonic Antigen | <7.7 | ng/mL |
| 65 | Human Chorionic Gonadotropin | <10 | mIU/mL |
| 66 | Prostate-Specific Antigen, Free | <4 | ng/mL |
| 67 | Prostate-Specific Antigen, Total | <8 | ng/mL |
| 68 | Prostatic Acid Phosphatase | 0.052-2.4 | ng/mL |
| 69 | Apolipoprotein A1 | 0.29-1.6 | mg/mL |
| 70 | Apolipoprotein CIII | 31-298 | ug/mL |
| 71 | Apolipoprotein H | 81-579 | ug/mL |
| 72 | B-type Natriuretic Peptide | <200 | pg/mL |
| 73 | Creatine Kinase MB | <8 | ng/mL |
| 74 | Endothelin 1 | <13 | pg/mL |
| 75 | Fatty Acid Binding Protein | <13 | ng/mL |
| 76 | Haptoglobin | <5 | mg/mL |
| 77 | Homocysteine | 5-15 | umol/L |
| 78 | Lipoprotein (a) | <537 | ug/mL |
| 79 | Myoglobin | 4.5-53 | ng/mL |
| 80 | Plasminogen Activator Inhibitor Type I | 8.3-207 | ng/mL |
| 81 | Pregnancy-Associated Plasma Protein A | <10 | mIU/mL |
| 82 | Vascular Cell Adhesion Molecule 1 | 354-1028 | ng/mL |
| 83 | Vascular Endothelial Growth Factor | 97-1222 | pg/mL |
| 84 | Brain-Derived Neurotrophic Factor | 0.17-49 | ng/mL |
| 85 | Eotaxin | <305 | pg/mL |
| 86 | Epidermal Growth Factor | <621 | pg/mL |
| 87 | Epithelial Neutrophil Activating Peptide 78 | <6.3 | ng/mL |
| 88 | Fibroblast Growth Factor-Basic Form | <617 | pg/mL |
| 89 | Granulocyte Macrophage Colony Stimulating | <63 | pg/mL |
| | Factor | | |
| 90 | Intercellular Adhesion Molecule 1 | 51-325 | ng/mL |
| 91 | Interleukin-1 Alpha | <0.44 | ng/mL |
| 92 | Interleukin-1 Beta | <9.9 | pg/mL |
| 93 | Interleukin-10 | <24 | pg/mL |
| 94 | Interleukin-l2p40 | <2.2 | ng/mL |
| 95 | Interleukin-l2p70 | <94 | pg/mL |
| 96 | Interleukin-13 | <126 | pg/mL |
| 97 | Interleukin-15 | <5 | ng/mL |
| 98 | Interleukin-16 | 195-1834 | pg/mL |
| 99 | Interleukin-18 | <800 | pg/mL |
| 100 | Interleukin-2 | <60 | pg/mL |
| 101 | Interleukin-3 | <2.5 | ng/mL |
| 102 | Interleukin-4 | <104 | pg/mL |
| 103 | Interleukin-5 | <48 | pg/mL |
| 104 | Interleukin-6 | <78 | pg/mL |
| 105 | Interleukin-7 | <82 | pg/mL |
| 106 | Interleukin-8 | <244 | pg/mL |
| 107 | Lymphotactin | <0.38 | ng/mL |
| 108 | Macrophage Inflammatory Protein 1 Alpha | <91 | pg/mL |
| 109 | Macrophage Inflammatory Protein 1 Beta | <891 | pg/mL |
| 110 | Macrophage-Derived Chemokine | 132-1123 | pg/mL |
| 111 | Matrix Metalloproteinase 2 | <337 | ng/mL |
| 112 | Matrix Metalloproteinase 3 | 1.6-55 | ng/mL |
| 113 | Matrix Metalloproteinase 9 | <436 | ng/mL |
| 114 | Monocyte Chemotactic Protein-1 | <1057 | pg/mL |
| 115 | Regulated Upon Activation, Normal T-cell | 0.76-91 | ng/mL |
| | Expressed and Secreted | | |
| 116 | Stem Cell Factor | <356 | pg/mL |
| 117 | Tissue Inhibitor of Metalloproteinase 1 | 63-420 | ng/mL |
| 118 | Tumor Necrosis Factor Alpha | <116 | pg/mL |
| 119 | Tumor Necrosis Factor Beta | <61 | pg/mL |
| 120 | Tumor Necrosis Factor Receptor 2 | 52-205 | ng/mL |
| 121 | Erythropoietin | <193 | pg/mL |
| 122 | Factor VII | 129-831 | ng/mL |
| 123 | Fibrinogen | <0.031 | mg/mL |
| 124 | Thrombopoietin | <6 | ng/mL |
| 125 | Tissue Factor | <5.3 | ng/mL |
| 126 | von Willebrand Factor | 1.8-54 | ug/mL |
| 127 | Adiponectin | 1.6-14 | ug/mL |
| 128 | Androstenedione | 0.6-3.3 | ng/mL |
| 129 | Cortisol | 2.5-25 | ug/dL |
| 130 | C-Peptide | 1-5 | ng/mL |
| 131 | Dihydroepiandrosterone Sulfate | 35-430 | ug/dL |
| 132 | Estradiol | <56 | pg/mL |
| 133 | Estriol, Unconjugated | <30 | ng/mL |
| 134 | Follicle Stimulating Hormone | 0.7-11.1 | mIU/mL |
| 135 | Gastrin | <115 | pg/mL |
| 136 | Growth Hormone | <7.4 | ng/mL |
| 137 | Insulin | <45 | uIU/mL |
| 138 | Insulin-Like Growth Factor Binding Protein 3 | 3.4-7.8 | ug/mL |
| 139 | Insulin-Like Growth Factor-1 | 70-358 | ng/mL |
| 140 | Leptin | 0.47-43 | ng/mL |
| 141 | Luteinizing Hormone | 0.1-200 | mIU/mL |
| 142 | Parathyroid Hormone | 16-87 | pg/mL |
| 143 | Progesterone | <1.1 | ng/mL |
| 144 | Prolactin | 0.6-29 | ng/mL |
| 145 | Sex Hormone-Binding Globulin | 13-180 | nmol/L |
| 146 | Testosterone | 20-1593 | ng/dL |
| 147 | Thyroglobulin Antigen | <55 | ng/mL |
| 148 | Thyroid Stimulating Hormone | 0.44-5.3 | uIU/mL |
| 149 | Thyroxine | 4.5-12.5 | ug/dL |
| 150 | Thyroxine Binding Globulin | 42-133 | ug/mL |
| 151 | Triiodothyronine | 80-200 | ngldL |
| 152 | Aspartate Aminotransferase Antigen | <16 | ug/mL |
| 153 | Complement 3 | 0.82-3 | mg/mL |
| 154 | C-Reactive Protein | 0.08-3 | ug/mL |
| 155 | Immunoglobulin A | 0.58-6.6 | mg/mL |
| 156 | Immunoglobulin E | <660 | ng/mL |
| 157 | Immunoglobulin M | 0.3-3.6 | mg/mL |
| 158 | Serum Amyloid P | 13-62 | ug/mL |
| 159 | Ferritin | 8.8-674 | ng/mL |
| 160 | Folic Acid | >3.5 | ng/mL |
| 161 | Vitamin B12 | 100-990 | pg/mL |
| 162 | Alpha-1-Antitrypsin | 1.1-3.1 | mg/mL |
| 163 | Alpha-2-Macroglobulin | 0.35-7.3 | mg/mL |
| 164 | Beta-2-Microglobulin | 1.2-5.3 | ug/mL |
| 165 | Glutathione S-Transferase | <135 | ng/mL |
| 166 | Rheumatoid Factor | NEGATIVE | |
| 167 | Anti-Nuclear Antibody | NEGATIVE | |
| 168 | Anti-Saccharomyces Cerevisiae Antibody | NEGATIVE | |
| 169 | Beta-2-Glycoprotein Antibody | NEGATIVE | |
| 170 | Centromere Protein B Antibody | NEGATIVE | |
| 171 | Collagen Type 1 Antibody | NEGATIVE | |
| 172 | Collagen Type 2 Antibody | NEGATIVE | |
| 173 | Collagen Type 4 Antibody | NEGATIVE | |
| 174 | Collagen Type 6 Antibody | NEGATIVE | |
| 175 | Complement Factor C1q Antibody | NEGATIVE | |
| 176 | Cytoebrome P450 Antibody | NEGATIVE | |
| 177 | Double-Stranded DNA Antibody | NEGATIVE | |
| 178 | Heat Shock Cognate Protein 70 Antibody | NEGATIVE | |
| 179 | Heat Shock Protein 32 Antibody | NEGATIVE | |
| 180 | Heat Shock Protein 65 Antibody | NEGATIVE | |
| 181 | Heat Shock Protein 71 Antibody | NEGATIVE | |
| 182 | Heat Shock Protein 90 Alpha Antibody | NEGATIVE | |
| 183 | Heat Shock Protein 90 Beta Antibody | NEGATIVE | |
| 184 | Histone Antibody | NEGATIVE | |
| 185 | Histone H1 Antibody | NEGATIVE | |
| 186 | Histone H2A Antibody | NEGATIVE | |
| 187 | Histone H2B Antibody | NEGATIVE | |
| 188 | Histone H3 Antibody | NEGATIVE | |
| 189 | Histone H4 Antibody | NEGATIVE | |
| 190 | Insulin Antibody | NEGATIVE | |
| 191 | JO-1 Antibody | NEGATIVE | |
| 192 | Mitochondrial Antibody | NEGATIVE | |
| 193 | Myeloperoxidase Antibody | NEGATIVE | |
| 194 | Pancreatic Islet Cell Antibody | NEGATIVE | |
| 195 | PM-1 Antibody | NEGATIVE | |
| 196 | Proliferating Cell Nuclear Antigen | NEGATIVE | |
| | Antibody | | |
| 197 | Proteinase 3 Antibody | NEGATIVE | |
| 198 | Ribosomal Nuclear Protein A Antibody | NEGATIVE | |
| 199 | Ribosomal Nuclear Protein Antibody | NEGATIVE | |
| 200 | Ribosomal Nuclear Protein C Antibody | NEGATIVE | |
| 201 | Ribosomal P Antibody | NEGATIVE | |
| 202 | Scleroderma 70 Antibody | NEGATIVE | |
| 203 | Smith Antibody | NEGATIVE | |
| 204 | SSA Antibody | NEGATIVE | |
| 205 | SSB Antibody | NEGATIVE | |
| 206 | Thyroglobulin Antibody | NEGATIVE | |
| 207 | Thyroid Microsomal Antibody | NEGATIVE | |
| 208 | Thyroxine Antibody | NEGATIVE | |
| 209 | Tissue Transglutaminase Antibody | NEGATIVE | |
| 210 | Triiodothyronine Antibody | NEGATIVE | |
| 211 | Adenovirus Antibody | NEGATIVE | |
| 212 | Bordetella pertussis Antibody | NEGATIVE | |
| 213 | Campylobacter jejuni Antibody | NEGATIVE | |
| 214 | Chlamydia pneumoniae Antibody | NEGATIVE | |
| 215 | Chlamydia trachomatis Antibody | NEGATIVE | |
| 216 | Cytomegalovirus Antibody | NEGATIVE | |
| 217 | Diphtheria Toxin Antibody | NEGATIVE | |
| 218 | Epstein-Barr Viral Capsid Antigen Antibody | NEGATIVE | |
| 219 | Epstein-Barr Virus Early Antigen Antibody | NEGATIVE | |
| 220 | Epstein-Barr Virus Nuclear Antigen Antibody | NEGATIVE | |
| 221 | Helicobacter pylori IgG Antibody | NEGATIVE | |
| 222 | Hepatitis B e Antibody | NEGATIVE | |
| 223 | Hepatitis D Antibody | NEGATIVE | |
| 224 | Hepatitis E Virus orf 2.3 kD Antibody | NEGATIVE | |
| 225 | Hepatitis E Virus orf 2.6 kD Antibody | NEGATIVE | |
| 226 | Hepatitis E Virus orf 3.3 kD Antibody | NEGATIVE | |
| 227 | Herpes Simplex Virus Type 1 Glycoprotein D | NEGATIVE | |
| | Antibody | | |
| 228 | Herpes Simplex Virus Type 2 Glycoprotein G | NEGATIVE | |
| | Antibody | | |
| 229 | Herpes Simplex Virus Types 1 and 2 | NEGATIVE | |
| | Antibody | | |
| 230 | Human Papilloma Virus Antibody | NEGATIVE | |
| 231 | Human T-cell Lymphotropic Virus Types | NEGATIVE | |
| | 1 and 2 Antibody | | |
| 232 | Influenza A Antibody | NEGATIVE | |
| 233 | Influenza A H3N2 Antibody | NEGATIVE | |
| 234 | Influenza B Antibody | NEGATIVE | |
| 235 | Leishmania donovani Antibody | NEGATIVE | |
| 236 | Lyme Disease Antibody | NEGATIVE | |
| 237 | Mumps Antibody | NEGATIVE | |
| 238 | Mycobacteria tuberculosis Antibody | NEGATIVE | |
| 239 | Mycoplasma pneumoniae Antibody | NEGATIVE | |
| 240 | Parainfluenza Type 1 Antibody | NEGATIVE | |
| 241 | Parainfluenza Type 2 Antibody | NEGATIVE | |
| 242 | Parainfluenza Type 3 Antibody | NEGATIVE | |
| 243 | Polio Antibody | NEGATIVE | |
| 244 | Respiratory Syncytial Virus Antibody | NEGATIVE | |
| 245 | Rubella Antibody | NEGATIVE | |
| 246 | Rubeola Antibody | NEGATIVE | |
| 247 | Streptolysin O Antibody | NEGATIVE | |
| 248 | Tetanus Toxin Antibody | NEGATIVE | |
| 249 | Toxoplasma Antibody | NEGATIVE | |
| 250 | Trypanosoma cruzi Antibody | NEGATIVE | |
| 251 | Varicella zoster IgG Antibody | NEGATIVE | |
| 252 | Varicella zoster 1gM Antibody | NEGATIVE | |

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. Also, the disclosure of these ranges is intended as a continuous range including every value between the minimum and maximum values.

A “normal” test result for each biomarker is typically defined to include biomarker levels that fall within the range of concentrations seen in normal, healthy individuals, as well as biomarker levels that fall within a set number of standard deviations of the range of concentrations seen in normal individuals. The preferred embodiment further comprises adjusting for each biomarker the standard deviation criteria that define a normal test result. For example and without limitation, where defining a normal test result as being within only one standard deviation of the range of concentrations seen in normal individuals would yield a number of positive indications that is too high based on the known incidence in the general population of a medical condition associated with the biomarker, the definition of a normal test result may be adjusted to include biomarker levels within two standard deviations of the range of concentrations seen in normal individuals, so as to reduce the number of false positives. The preferred embodiment also comprises adjusting for each biomarker the standard deviation criteria that define a normal test result such that the non-incorporated results does not have to be equally divided between above the range and below the range.

In another embodiment, a “normal” test is defined with respect to changes in serial results, as discussed below. In this case, a “normal” test result for each biomarker can be defined to include biomarker levels that do not necessarily fall within the range of concentrations seen in normal, healthy individuals, as well as biomarker levels that fall within a set number of standard deviations of the range of concentrations seen in normal individuals. A significant change in serial results in a level of a biomarker can indicate a positive diagnosis of a condition or that more investigation is needed, even though the level of the biomarker is within a range of concentrations seen in normal, healthy individuals.

Reference Change Values

Changes in serial results from an individual can occur because of pre-analytical, analytical, within-subject biological variation, and changes in a condition of a patient. Thus, if pre-analytical variation can be minimized and the changes in a patient's health status are taken into consideration, then to assess a condition of a patient, a change in a condition, such as change in levels of biomarkers over time, can be compared to the variation due to analytical variation (CV_{A}) and within-subject biological variation (CV_{W}). Analytical variation (CV_{A}) and within-subject biological variation (CV_{W}) can be calculated into a reference change value (RCV). Thus, RCV can identify significant changes in the state of patients when screening with Health Assessment Service or monitoring a known condition. By monitoring serial results and calculating RCV, a practitioner can determine if an unknown condition or disease may be developing or a known condition is improving or deteriorating.

Pre-analytic variation occurs before the analytical phase of generation of observed value. The sources of variation can be divided into two types: factors that affect the individual before specimen collection occurs and factors inherent in the collection and handling of the specimens. Pre-analytic variation can be minimized by adoption of strict protocols for sample patient preparation, and sample collection, transport, and handling.

CV_{A} is analytic precision obtained from internal quality control at the appropriate clinical decision making level and is commonly available for analytes in laboratories. Analytical variation can be expressed as the weighted mean of variances from the data. After obtaining raw data, CV_{A} can be calculated with analysis of variance. Optionally, presence of outliers in the raw data is evaluated before analysis of variance is applied to calculate analytical variation. Analytical variation can be minimized by setting internal quality control and evaluation of laboratory performance. Analytical characteristics that are taken into account are imprecision and change in bias. Imprecision is random error and is defined as the closeness of agreement between independent results of measurements obtained under stipulated conditions. In practice, imprecision is determined by replicate analysis and the dispersion calculated as standard deviation (SD) or coefficient of variation (CV). Bias is systematic error and is defined as the difference between the expectation of measurement results and the true value. In practice, bias is the difference between observed values and some estimate of the true value. Changes in bias can be a source of variation in serial results: these can be caused by re-calibration of methodology, lot-to-lot variation in commercial calibrants, and lot-to-lot variation in reagents. This source of random systematic error is usually accounted for in longer-term estimates of imprecision calculated over a number of calibration cycles and thus will be considered negligible.

CV_{W} is mean within-subject biological variation that can be calculated for certain conditions or can be taken from comprehensive literature. (Fraser CGF (2001) Biological variation: from principles to practice. AACC Press, Washington D.C.; Ricos C, et al. (1999) Scand J. Clin Lab Invest 59:491; Ricos C, et al. http://westgard.com/guest17.htm; all of which are herein incorporated by reference in their entirety.) After obtaining raw data, CV_{W} can be calculated with analysis of variance. Optionally, presence of outliers in the raw data is evaluated before analysis of variance is applied to calculate within subject variation. Biological variation can be described as random around a homeostatic setting point. Data for biological variation can be generated with more than one sample obtained from each of a small cohort of subjects rather than a single sample obtained from a large number of subjects.

To assess a condition of a patient, a reference change value (RCV) is calculated with the following equation: RCV=2^{1/2}Z(CV_{A}²+CV_{W}²)^{1/2} and the RCV is compared to the change in the condition. In the formula, 2^{1/2} is 1.414. Bidirectional Z score is found in standard statistical tables. Most often, 1.96 is used as significant, that is, P<0.05 (95% confidence level), and 2.58 is used for highly significant, that is, P<0.01 (99% confidence level). Other values can be used for bidirectional Z score aligned with other confidence values.

The RCVs can be used to point out results on reports and to invoke verification by professionals. Thus, a “normal” test result for each biomarker can be defined with respect to RCV. A significant change in serial results in a level of a biomarker can indicate a positive diagnosis of a condition or that more investigation is needed, even though the level of the biomarker is within a range of concentrations seen in normal, healthy individuals.

Accordingly, embodiments provide for a method of producing a reference change value for a biomarker comprising

(a) obtaining levels of the biomarker from a population of at least 20 healthy individuals at least five times from each individual;

(b) using the levels obtained from (a) to determine mean within-subject biological variation (CV_{W});

(c) determining an analytic precision (CV_{A});

(d) determining a bidirectional Z score for desired level of confidence;

(e) calculating a reference change value (RCV) for the population, wherein RCV=2^{1/2}Z(CV_{A}²+CV_{W}²)^{1/2}, wherein Z is standard deviate appropriate for chosen probability, CV_{A} is analytic precision, CV_{W} is mean within-subject biological variation.

Another embodiment provides for a method of diagnosing a condition in a patient comprising

(a) measuring levels of biomarkers in a test panel comprising one or more of biomarkers in a first sample from a patient at a first time,
wherein the biomarkers are associated with the condition;

(b) measuring levels of biomarkers in a second sample from the patient at a second time, wherein the biomarkers in (b) are the same as the biomarkers in (a);

(c) calculating a percentage change between the levels of biomarkers in (a) and (b); and

(d) comparing the percentage change in (c) to a reference change value; wherein
(i) percentage change in (c) that is equal to or higher than the reference change value indicates a possibility of a presence of the condition in the patient; or
(ii) percentage change in (c) that is lower than the reference change value indicates a decreased possibility of a presence of the condition in the patient.
   In certain cases, a possibility of a presence of the condition is a positive diagnosis of the condition. In certain cases, a decreased possibility of a presence of the condition is a negative diagnosis of the condition.

In certain embodiments, the condition is selected from the group consisting of autoimmunity, cardiovascular disease, cancer, cell signaling, diabetes, endocrine function, hematology, immunity/inflammation, infectious disease, nutrition, organ system function, and osteoarthritis.

Data on the components of variation can be generated in a number of ways (Fraser C G, Harris E K, Generation and Application of Data on Biological Variation in Clinical Chemistry. Crit. Rev. Clin. Lab. Sci. 1989; 27:409-37, herein incorporated by reference in its entirety.) In calculating RCV, a number of specimens is collected from each of a small cohort of individuals rather than one specimen being collected from a large reference sample group. For example:
a small number of reference individuals is chosen;
ideally, individuals who are not ostensibly healthy or who have an unconventional diet or lifestyle, take over the counter medications or prescribed drugs, or overuse alcohol or other recreational materials are excluded, although it is difficult to ensure that these exclusion criteria are applied correctly since individuals may not be totally open about their lifestyles;
a series of specimens is collected from each individual while taking care to minimize pre-analytic variation through, for example, for serum specimens, taking all at the same time of day, using one phlebotomist and one batch of tubes, with very standard operating procedures for specimen collection, transport, and handling;
specimens are then stored in conditions that ensure stability;
when specimens have been collected, they are prepared for examination by thawing and mixing for example, and then they are examined in random duplicate in a single analytic batch;
then the data is assessed for outliers; and
since the contribution of pre-analytical variation has been minimized, nested analysis of variance is undertaken to generate estimates of the components, namely, analytic variation and within-subject biological variation.

Table 2 shows within-subject biological variation of certain analytes. These values can be used to calculate RCVs.

TABLE 2

| | | CV-w |
|---|---|---|
| No. | Analyte | (in %) |
| 1 | Glucose | 6.5 |
| 2 | Blood Urea Nitrogen | 13.7 |
| 3 | Creatinine | 6.3 |
| 4 | Uric Acid | 8.6 |
| 5 | Sodium | 0.8 |
| 6 | Potassium | 4.8 |
| 7 | Chloride | 1.3 |
| 8 | Calcium | 2.3 |
| 9 | Phosphorus | 8.5 |
| 10 | Magnesium | 4.1 |
| 11 | Cholesterol | 6.0 |
| 12 | Triglycerides | 22.0 |
| 13 | High Density Lipoprotein Cholesterol | 7.5 |
| 14 | Low Density Lipoprotein Cholesterol | 8.6 |
| 15 | Total Protein | 2.7 |
| 16 | Albumin | 3.3 |
| 17 | Globulin | 5.5 |
| 18 | Bilirubin, Total | 25.6 |
| 19 | Alkaline Phosphatase | 9.1 |
| 20 | Gamma Glutamyl Transferase | 13.8 |
| 21 | Aspartate Aminotransferase | 11.9 |
| 22 | Alanine Aminotransferase | 24.3 |
| 23 | Lactate Dehydrogenase | 12.9 |
| 24 | Creatine Kinase, Total | 31.7 |
| 25 | Amylase | 11.1 |
| 26 | Iron, Serum | 26.6 |
| 27 | White Blood Cell Count | 11.2 |
| 28 | Red Blood Cell Count | 3.2 |
| 29 | Hemoglobin | 3.4 |
| 30 | Hematocrit | 2.8 |
| 31 | Mean Corpuscular Volume | 1.3 |
| 32 | Mean Corpuscular Hemoglobin | 1.6 |
| 33 | Mean Corpuscular Hemoglobin Concentration | 1.7 |
| 34 | Red Cell Distribution Width | 3.5 |
| 35 | Neutrophil Count | 23.6 |
| 36 | Lymphocyte Count | 12.3 |
| 37 | Eosinophil Count | 21.0 |
| 38 | Basophil Count | 28.0 |
| 39 | Platelet Count | 9.1 |
| 40 | Hemoglobin A1c | 8.8 |
| 41 | Cancer Antigen 125 | 36.0 |
| 42 | Cancer Antigen 15-3 (BR-MA) | 5.7 |
| 43 | Cancer Antigen 19-9 | 24.5 |
| 44 | Carcinoembryonic Antigen | 11.9 |
| 45 | Prostate-Specific Antigen, Total | 18.1 |
| 46 | Apolipoprotein A1 | 7.5 |
| 47 | Creatine Kinase MB | 18.4 |
| 48 | Haptoglobin | 23.3 |
| 49 | Homocysteine | 7.7 |
| 50 | Lipoprotein (a) | 10.8 |
| 51 | Myoglobin | 13.9 |
| 52 | von Willebrand Factor | 0.001 |
| 53 | Androstenedione | 15.8 |
| 54 | Cortisol | 20.9 |
| 55 | C-Peptide | 9.3 |
| 56 | Dihydroepiandrosterone Sulfate | 3.4 |
| 57 | Estradiol | 41.6 |
| 58 | Follicle Stimulating Hormone | 10.1 |
| 59 | Insulin | 21.1 |
| 60 | Luteinizing Hormone | 14.5 |
| 61 | Progesterone | 31.3 |
| 62 | Prolactin | 23.7 |
| 63 | Sex Hormone-Binding Globulin | 12.1 |
| 64 | Testosterone | 9.6 |
| 65 | Thyroglobulin Antigen | 13.0 |
| 66 | Thyroid Stimulating Hormone | 20.0 |
| 67 | Thyroxine (T4) | 6.0 |
| 68 | Thyroxine Binding Globulin | 6.0 |
| 69 | Triiodothyronine (T3) | 8.7 |
| 70 | Complement 3 | 5.2 |
| 71 | C-Reactive Protein | 56.6 |
| 72 | Immunoglobulin A | 6.8 |
| 73 | Immunoglobulin M | 7.6 |
| 74 | Ferritin | 12.8 |
| 75 | Alpha-1-Antitrypsin | 4.8 |
| 76 | Alpha-2-Macroglobulin | 3.3 |
| 77 | Beta-2-Microglobulin | 4.4 |
| | CV_{w} for the following biomarkers are to | (tbd: to be |
| | be determined | determined) |
| 78 | Plasminogen Activator Inhibitor Type I | tbd |
| 79 | Pregnancy-Associated Plasma Protein A | tbd |
| 80 | Vascular Cell Adhesion Molecule 1 | tbd |
| 81 | Vascular Endothelial Growth Factor | tbd |
| 82 | Brain-Derived Neurotrophic Factor | tbd |
| 83 | Eotaxin | tbd |
| 84 | Epidermal Growth Factor | tbd |
| 85 | Epithelial Neutrophil Activating Peptide 78 | tbd |
| 86 | Fibroblast Growth Factor-Basic Form | tbd |
| 87 | Granulocyte Macrophage Colony Stimulating Factor | tbd |
| 88 | Intercellular Adhesion Molecule 1 | tbd |
| 89 | Interleukin-1 Alpha | tbd |
| 90 | Interleukin-1 Beta | tbd |
| 91 | Interleukin-10 | tbd |
| 92 | Interleukin-12p40 | tbd |
| 93 | Interleukin-12p70 | tbd |
| 94 | Interleukin-13 | tbd |
| 95 | Interleukin-15 | tbd |
| 96 | Interleukin-16 | tbd |
| 97 | Interleukin-18 | tbd |
| 98 | Interleukin-2 | tbd |
| 99 | Interleukin-3 | tbd |
| 100 | Interleukin-4 | tbd |
| 101 | Interleukin-5 | tbd |
| 102 | Interleukin-6 | tbd |
| 103 | Interleukin-7 | tbd |
| 104 | Interleukin-8 | tbd |
| 105 | Lymphotactin | tbd |
| 106 | Macrophage Inflammatory Protein 1 Alpha | tbd |
| 107 | Macrophage Inflammatory Protein 1 Beta | tbd |
| 108 | Macrophage-Derived Chemokine | tbd |
| 109 | Matrix Metalloproteinase 2 | tbd |
| 110 | Matrix Metalloproteinase 3 | tbd |
| 111 | Matrix Metalloproteinase 9 | tbd |
| 112 | Monocyte Chemotactic Protein-1 | tbd |
| 113 | Regulated Upon Activation, Normal T-cell | tbd |
| | Expressed and Secreted | |
| 114 | Stem Cell Factor | tbd |
| 115 | Tissue Inhibitor of Metalloproteinase 1 | tbd |
| 116 | Tumor Necrosis Factor Alpha | tbd |
| 117 | Tumor Necrosis Factor Beta | tbd |
| 118 | Tumor Necrosis Factor Receptor 2 | tbd |
| 119 | Erythropoietin | tbd |
| 120 | Factor VII | tbd |
| 121 | Fibrinogen | tbd |
| 122 | Thrombopoietin | tbd |
| 123 | Tissue Factor | tbd |
| 124 | Adiponectin | tbd |
| 125 | Estriol, Unconjugated | tbd |
| 126 | Gastrin | tbd |
| 127 | Growth Hormone | tbd |
| 128 | Insulin-Like Growth Factor Binding Protein 3 | tbd |
| 129 | Insulin-Like Growth Factor-1 | tbd |
| 130 | Leptin | tbd |
| 131 | Parathyroid Hormone | tbd |
| 132 | Aspartate Aminotransferase Antigen | tbd |
| 133 | Immunoglobulin E | tbd |
| 134 | Serum Amyloid P | tbd |
| 135 | Folic Acid | tbd |
| 136 | Vitamin B12 | tbd |
| 137 | Glutathione S-Transferase | tbd |
| 138 | Rheumatoid Factor | tbd |
| 139 | Anti-Nuclear Antibody | tbd |
| 140 | Anti-Saccharomyces Cerevisiae Antibody | tbd |
| 141 | Beta-2-Glycoprotein Antibody | tbd |
| 142 | Centromere Protein B Antibody | tbd |
| 143 | Collagen Type 1 Antibody | tbd |
| 144 | Collagen Type 2 Antibody | tbd |
| 145 | Collagen Type 4 Antibody | tbd |
| 146 | Collagen Type 6 Antibody | tbd |
| 147 | Complement Factor C1q Antibody | tbd |
| 148 | Cytochrome P450 Antibody | tbd |
| 149 | Double-Stranded DNA Antibody | tbd |
| 150 | Heat Shock Cognate Protein 70 Antibody | tbd |
| 151 | Heat Shock Protein 32 Antibody | tbd |
| 152 | Heat Shock Protein 65 Antibody | tbd |
| 153 | Heat Shock Protein 71 Antibody | tbd |
| 154 | Heat Shock Protein 90 Alpha Antibody | tbd |
| 155 | Heat Shock Protein 90 Beta Antibody | tbd |
| 156 | Histone Antibody | tbd |
| 157 | Histone H1 Antibody | tbd |
| 158 | Histone H2A Antibody | tbd |
| 159 | Histone H2B Antibody | tbd |
| 160 | Histone H3 Antibody | tbd |
| 161 | Histone H4 Antibody | tbd |
| 162 | Insulin Antibody | tbd |
| 163 | JO-1 Antibody | tbd |
| 164 | Mitochondrial Antibody | tbd |
| 165 | Myeloperoxidase Antibody | tbd |
| 166 | Pancreatic Islet Cell Antibody | tbd |
| 167 | PM-1 Antibody | tbd |
| 168 | Proliferating Cell Nuclear Antigen Antibody | tbd |
| 169 | Proteinase 3 Antibody | tbd |
| 170 | Ribosomal Nuclear Protein A Antibody | tbd |
| 171 | Ribosomal Nuclear Protein Antibody | tbd |
| 172 | Ribosomal Nuclear Protein C Antibody | tbd |
| 173 | Ribosomal P Antibody | tbd |
| 174 | Scleroderma 70 Antibody | tbd |
| 175 | Smith Antibody | tbd |
| 176 | SSA Antibody | tbd |
| 177 | SSB Antibody | tbd |
| 178 | Thyroglobulin Antibody | tbd |
| 179 | Thyroid Microsomal Antibody | tbd |
| 180 | Thyroxine Antibody | tbd |
| 181 | Tissue Transglutaminase Antibody | tbd |
| 182 | Triiodothyronine Antibody | tbd |
| 183 | Adenovirus Antibody | tbd |
| 184 | Bordetella pertussis Antibody | tbd |
| 185 | Campylobacter jejuni Antibody | tbd |
| 186 | Chlamydia pneumoniae Antibody | tbd |
| 187 | Chlamydia trachomatis Antibody | tbd |
| 188 | Cytomegalovirus Antibody | tbd |
| 189 | Diphtheria Toxin Antibody | tbd |
| 190 | Epstein-Barr Viral Capsid Antigen Antibody | tbd |
| 191 | Epstein-Barr Virus Early Antigen Antibody | tbd |
| 192 | Epstein-Barr Virus Nuclear Antigen Antibody | tbd |
| 193 | Helicobacter pylori IgG Antibody | tbd |
| 194 | Hepatitis B e Antibody | tbd |
| 195 | Hepatitis D Antibody | tbd |
| 196 | Hepatitis E Virus orf 2.3 kD Antibody | tbd |
| 197 | Hepatitis E Virus orf 2.6 kD Antibody | tbd |
| 198 | Hepatitis E Virus orf 3.3 kD Antibody | tbd |
| 199 | Herpes Simplex Virus Type 1 Glycoprotein D | tbd |
| | Antibody | |
| 200 | Herpes Simplex Virus Type 2 Glycoprotein G | tbd |
| | Antibody | |
| 201 | Herpes Simplex Virus Types 1 and 2 Antibody | tbd |
| 202 | Human Papilloma Virus Antibody | tbd |
| 203 | Human T-cell Lymphotropic Virus Types 1 and 2 | tbd |
| | Antibody | |
| 204 | Influenza A Antibody | tbd |
| 205 | Influenza A H3N2 Antibody | tbd |
| 206 | Influenza B Antibody | tbd |
| 207 | Leishmania donovani Antibody | tbd |
| 208 | Lyme Disease Antibody | tbd |
| 209 | Mumps Antibody | tbd |
| 210 | Mycobacteria tuberculosis Antibody | tbd |
| 211 | Mycoplasma pneumoniae Antibody | tbd |
| 212 | Parainfluenza Type 1 Antibody | tbd |
| 213 | Parainfluenza Type 2 Antibody | tbd |
| 214 | Parainfluenza Type 3 Antibody | tbd |
| 215 | Polio Antibody | tbd |
| 216 | Respiratory Syncytial Virus Antibody | tbd |
| 217 | Rubella Antibody | tbd |
| 218 | Rubeola Antibody | tbd |
| 219 | Streptolysin O Antibody | tbd |
| 220 | Tetanus Toxin Antibody | tbd |
| 221 | Toxoplasma Antibody | tbd |
| 222 | Trypanosoma cruzi Antibody | tbd |
| 223 | Varicella zoster IgG Antibody | tbd |
| 224 | Varicella zoster IgM Antibody | tbd |
| 225 | Blood Urea Nitrogen/Creatinine Ratio | tbd |
| 226 | Carbon Dioxide | tbd |
| 227 | Low Density Lipoprotein/High Density Lipoprotein | tbd |
| | Ratio | |
| 228 | Albumin/Globulin Ratio | tbd |
| 229 | Iron Binding Capacity, Unsaturated | tbd |
| 230 | Iron Binding Capacity, Total | tbd |
| 231 | Iron, Percent Saturated | tbd |
| 232 | Neutrophil Percentage | tbd |
| 233 | Lymphocyte Percentage | tbd |
| 234 | Monocyte Count | tbd |
| 235 | Monocyte Percentage | tbd |
| 236 | Eosinophil Percentage | tbd |
| 237 | Basophil Percentage | tbd |
| 238 | Hepatitis A Antibody | tbd |
| 239 | Hepatitis B Core Antibody | tbd |
| 240 | Hepatitis B Surface Antigen | tbd |
| 241 | Hepatitis B Surface Antibody | tbd |
| 242 | Hepatitis C Antibody | tbd |
| 243 | Alpha Fetoprotein | tbd |
| 244 | Calcitonin | tbd |
| 245 | Human Chorionic Gonadotropin | tbd |
| 246 | Prostate-Specific Antigen, Free | tbd |
| 247 | Prostatic Acid Phosphatase | tbd |
| 248 | Apolipoprotein CIII | tbd |
| 249 | Apolipoprotein H | tbd |
| 250 | B-type Natriuretic Peptide | tbd |
| 251 | Endothelin 1 | tbd |
| 252 | Fatty Acid Binding Protein | tbd |

Conditions

Biomarkers are naturally occurring substances that can signal disease or indicate conditions when found at concentrations different from levels in normal healthy individuals or change significantly within an individual. The biomarkers in Tables 1 and 2 can be grouped into panels based on their association with certain diseases and organ system functions. These panels include biomarkers specific for conditions such as autoimmunity, cardiovascular disease, cancer, cell signaling, diabetes, endocrine function, hematology, immunity/inflammation, infectious disease, nutrition, organ system function, and osteoarthritis. In an embodiment, a panel for a condition comprises at least 1 biomarker associated with the condition. In certain embodiments, a panel for a condition comprises at least 2, 2 or more, 5, 5 or more, 10, or 10 or more biomarkers associated with the condition.

In one embodiment, to diagnose a condition, a practitioner runs a panel for the condition with at least one biomarker associated with the condition resulting in positive diagnosis for the biomarker. In another embodiment, to diagnose a condition, a practitioner runs a panel for the condition with at least 2 biomarkers associated with the condition resulting in positive diagnosis for one or two biomarkers. In another embodiment, to diagnose a condition, a practitioner runs a panel for the condition with at least 5 biomarkers associated with the condition resulting in positive diagnosis for 1, 2, 3, 4, or 5 biomarkers. In another embodiment, to diagnose a condition, a practitioner runs a panel for the condition with at least 10 biomarkers associated with the condition resulting in positive diagnosis for 1, 2, 3, 4, 5, 6, 7, 8, 9, or 10 biomarkers.

Biomarker

anti-nuclear antibody
C-reactive protein
double-stranded DNA antibody
ferritin
haptoglobulin
rheumatoid factor
beta-2-glycoprotein
centromere protein B antibody
collagen type 6 antibody
complement factor C1Q antibody
histone antibody
histone H1 antibody
histone H2A antibody
histone H2B antibody
histone H3 antibody
histone H4 antibody
JO-1 antibody
myeloperoxidase antibody
PM-1 antibody
proliferating cell nuclear antigen antibody
proteinase 3 antibody
ribosomal nuclear protein antibody
ribosomal nuclear protein A antibody
ribosomal nuclear protein C antibody
ribosomal P antibody
scleroderma 70 antibody
smith antibody
SSA antibody
SSB antibody
basophil count
basophil percentage
beta-2 microglobulin
cancer antigen 125
carcinoembryonic antigen
dihydroepiandrosterone sulfate
eosinophil count
eosinophil percentage
erythropoietin
follicle stimulating hormone
globulin
growth hormone
haptoglobin
Helicobacter pylori IgG antibody
hematocrit
hemoglobin
hepatitis C antibody
human chorionic gonadotropin
immunoglobulin A
immunoglobulin M
insulin
lactate dehydrogenase
luteinizing hormone
lymphocyte count
lymphocyte percentage
monocyte count
monocyte percentage
neutrophil count
neutrophil percentage
platelet count
prolactin
prostate-specific antigen-free
prostate specific antigen-total
testosterone
total protein
white blood cell count
alpha fetoprotein
calcitonin
cancer antigen 15-3
cancer antigen 19-9
endothelin 1
Factor VII
gastrin
glutathione S-transferase
human papilloma virus antibody
prostatic acid phosphatase

Biomarker

C-reactive protein
cholesterol
creatine kinase MB
creatine kinase-total
ferritin
fibrinogen
haptoglobin
high-density lipoprotein
homocysteine
low-density lipoprotein
low-density lipoprotein/high-density lipoprotein ratio
triglycerides
von Willebrand factor
apolipoprotein A1
B-type natriuretic peptide
endothelin 1
lipoprotein (a)
myeloperoxidase antibody
myoglobin
plasminogen activator inhibitor type 1
proliferating cell nuclear antigen antibody
proteinase 3 antibody
apolipoprotein CIII
apolipoprotein H
fatty acid binding protein
fibroblast growth factor-basic form
heat shock cognate protein 70 antibody
heat shock protein 32 antibody
heat shock protein 66 antibody
heat shock protein 71 antibody
heat shock protein 90 alpha antibody
heat shock protein 90 beta antibody
pregnancy-associated plasma protein A

Biomarker

brain-derived neurotrophic factor
eotaxin
epidermal growth factor
fibroblast growth factor-basic form
granulocyte macrophage colony stimulating factor
insulin-like growth factor binding protein 3
insulin-like growth factor 1
intercellular adhesion molecule 1
interleukin-1 alpha
interleukin-1 beta
interleukin-2
interleukin-3
interleukin-4
interleukin-5
interleukin-6
interleukin-7
interleukin-8
interleukin-10
interleukin-12 p40
interleukin-12 p70
interleukin-13
interleukin-15
interleukin-16
interleukin-18
lymphotactin
macrophage-derived chemokine
macrophage inflammatory protein 1 alpha
macrophage inflammatory protein 1-beta
matrix metalloproteinase 2
matrix metalloprotein-3
matrix, metalloprotein-9
monocyte chemotactic protein-1
regulated upon activation
normal T-cell expressed and secreted
stem cell factor
tissue inhibitor of metalloproteinase 1
tumor necrosis factor alpha
tumor necrosis factor beta
tumor necrosis factor receptor 2
vascular cell adhesion molecule 1
vascular endothelial growth factor

Biomarker

Glucose
Insulin
insulin antibody
C-peptide
hemoglobin A1c
leptin
pancreatic islet cell antibody
adiponectin
insulin-like growth factor binding protein 3
insulin-like growth factor-1

Biomarker

cortisol
follicle stimulating hormone
growth hormone
luteinizing hormone
prolactin
thyroid stimulating hormone
calcium
parathyroid hormone
phosphorus
thyroglobulin antibody
thyroid microsomal antibody
thyroid stimulating hormone
thyroxine
thyroxine binding globulin
triiodothyronine
calcitonin
thyroglobulin antigen
thyroxine antibody
triiodothyronine antibody
dihydroepiandrosterone sulfate
estradiol
follicle stimulating hormone
luteinizing hormone
progesterone
prolactin
testosterone
androstenedione
estriol
unconjugated
sex hormone-binding globulin

Biomarker

bilirubin-total
eosinophil count
eosinophil percentage
erythropoietin
ferritin
fibrinogen
hematocrit
hemoglobin
iron binding capacity-total
iron-serum
lactate dehydrogenase
lymphocyte count
lymphocyte percentage
mean corpuscular hemoglobin
mean corpuscular hemoglobin concentration
mean corpuscular volume
monocyte count
monocyte percentage
platelet count
red blood cell count
red cell distribution width
vitamin B-12
white blood cell count
basophil count
basophil percentage
Factor VII
haptoglobin
thromobopoietin
tissue factor
von Willebrand factor

Biomarker

aspartate aminotransferase antigen
C-reactive protein
haptoglobin
immunoglobulin A
immunoglobulin E
immunoglobulin M
von Willebrand factor
Factor VII
alpha-2-macroglobulin
complement-3
epithelial neutrophil activating peptide 78
heat shock cognate protein 70 antibody
heat shock protein 32 antibody
heat shock protein 65 antibody
heat shock protein 71 antibody
heat shock protein 90 alpha antibody
heat shock protein 90 beta antibody
serum amyloid P

Biomarker

Helicobacter pylori IgG antibody
Mycoplasma pneumoniae antibody
Streptolysin O antibody
Bordetella pertussis antibody
Campylobacter jejuni antibody
Chlamydia pneumoniae antibody
Chlamydia trachomatis antibody
Diphtheria toxin antibody
Leishmania donovani antibody
Lyme disease antibody
Mycobacteria tuberculosis antibody
Tetanus antibody
Toxoplasma gondi antibody
Trypanosoma cruzi antibody
Cytomegalovirus antibody
Epstein-Barr virus early antigen antibody
Hepatitis A antibody
Hepatitis B core antibody
Hepatitis B e antibody
Hepatitis B surface antibody
Hepatitis B surface antigen
Hepatitis C antibody
Hepatitis D antibody
Hepatitis E orf 2.3 kD antibody
Hepatitis orf 2.6 kD antibody
Hepatitis orf 3.3 kD antibody
Influenza A H3N2 antibody
Rubella antibody
Rubeola antibody
Varicella zoster IgG antibody
Varicella zoster IgM antibody
Adenovirus antibody
Herpes simplex virus type 1 glycoprotein D antibody
Herpes simplex virus type 2 glycoprotein G antibody
Herpes simplex virus types 1 and 2 antibodies
human papilloma virus antibody
human T-cell lymphotropic virus types 1 and 2 antibodies
influenza A antibody
influenza B antibody
mumps antibody
parainfluenza type 1 antibody
parainfluenza type 2 antibody
parainfluenza type 3 antibody
polio antibody
respiratory syncytial virus antibody
Epstein-Barr nuclear antigen antibody
Epstein-Barr viral capside antigen antibody

Biomarker

albumin
albumin/globulin ratio
amylase
calcium
carbon dioxide
chloride
cholesterol
ferritin
folic acid
globulin
glucose
hematocrit
hemoglobin
iron binding capacity-total
iron binding capacity-unsaturated
iron-percent saturated
iron-serum
magnesium
mean corpuscular hemoglobin
mean corpuscular hemoglobin concentration
phosphorus
potassium
sodium
total protein
triglycerides
uric acid
vitamin B-12

Biomarker

Helicobacter pylori IgG antibody
Campylobacter jejuni antibody
anti-Saccharomyces cerevisiae antibody
gastin
tissue transglutaminase antibody
blood urea nitrogen
blood urea nitrogen/creatinine ratio
carbon dioxide
chloride
creatinine
potassium
sodium
uric acid
beta-2-microglobulin
alanine aminotransferase
alkaline phosphatase
aspartate aminotransferase
bilirubin-total
ferritin
fibrinogen
gamma glutamyl transferase
haptoglobin
hepatitis A antibody
hepatitis B core antibody
hepatitis B e antibody
hepatitis B surface antibody
hepatitis B surface antigen
hepatitis C antibody
hepatitis D antibody
hepatitis E orf 2.3 antibody
hepatitis E orf 2.6 antibody
hepatitis E orf 3.3 antibody
iron binding capacity-total
lactate dehydrogenase
alpha-1-antitrypsin
cytochrome P-450 antibody
glutathione S-transferase
mitochondrial antibody

Biomarker

C-reactive protein
ferritin
haptoglobin
rheumatoid factor
von Willebrand factor
anti-nuclear antibody
collagen type 1 antibody
collagen type 2 antibody
collagen type 4 antibody
collagen type 6 antibody
heat shock cognate protein 70 antibody
heat shock protein 32 antibody
heat shock protein 65 antibody
heat shock protein 71 antibody
heat shock protein 90 alpha antibody
heat shock protein 90 beta antibody

Administration and Application

All patents referenced herein are incorporated by reference. Analyte levels can be measured using an immunoassay such as an ELISA or a multiplexed method as described below, and in more detail by Chandler et al., U.S. Pat. No. 5,981,180 (Luminex Corporation). The analytes used in the method of the invention can be detected, for example, by a binding assay. The term “binding reagent” and like terms, refers to any compound, composition or molecule capable of specifically or substantially specifically (that is with limited cross-reactivity) binding another compound or molecule, which, in the case of immune-recognition is an epitope. The binding reagents typically are antibodies, preferably monoclonal antibodies, or derivatives or analogs thereof, but also include, without limitation: Fv fragments; single chain Fv (scFv) fragments; Fab′ fragments; F(ab′)2 fragments; humanized antibodies and antibody fragments; camelized antibodies and antibody fragments; and multivalent versions of the foregoing. Multivalent binding reagents also may be used, as appropriate, including without limitation: monospecific or bispecific antibodies, such as disulfide stabilized Fv fragments, scFv tandems ((scFv)2 fragments), diabodies, tribodies or tetrabodies, which typically are covalently linked or otherwise stabilized (i.e., leucine zipper or helix stabilized) scFv fragments. “Binding reagents” also include aptamers, as are described in the art.

Methods of making antigen-specific binding reagents, including antibodies and their derivatives and analogs and aptamers, are well-known in the art. Polyclonal antibodies can be generated by immunization of an animal. Monoclonal antibodies can be prepared according to standard (hybridoma) methodology. Antibody derivatives and analogs, including humanized antibodies can be prepared recombinantly by isolating a DNA fragment from DNA encoding a monoclonal antibody and subcloning the appropriate V regions into an appropriate expression vector according to standard methods. Phage display and aptamer technology are described in the literature and permit in vitro clonal amplification of antigen-specific binding reagents with very low affinity and cross-reactivity. Phage display reagents and systems are available commercially, and include the Recombinant Phage Antibody System (RPAS), commercially available from Amersham Pharmacia Biotech, Inc. of Piscataway, N.J. and the pSKAN Phagemid Display System, commercially available from MoBiTec, LLC of Marco Island, Fla. Aptamer technology is described for example and without limitation in U.S. Pat. Nos. 5,270,163, 5,475096, 5,840867, and 6,544,776.

The ELISA and Luminex LabMAP immunoassays described below are examples of sandwich assays. As used herein, “immunoassays” refer to immune assays, typically, but not exclusively to any one assay, capable of detecting and quantifying a desired blood biomarker, namely at least one of the analytes listed in Table 1, or any combination thereof. The term “sandwich assay” refers to an immunoassay where the antigen is sandwiched between two binding reagents, which are typically antibodies—the first binding reagent/antibody being attached to a surface and the second binding reagent/antibody comprising a detectable group. Examples of detectable groups include, for example and without limitation: fluorochromes, enzymes, or epitopes for binding an additional binding reagent (for example, when the second binding reagent/antibody is a mouse antibody, such an epitope is detectable by an additional fluorescently labeled anti-mouse antibody), such as an antigen or member of a binding pair, such as biotin. The surface may be a planar surface, such as in the case of a typical grid-type array (for example, but without limitation, 96-well plates and planar microarrays), as described herein, or a non-planar surface, as with coated bead array technologies, where each “species” of bead is labeled with, for example, a fluorochrome (such as the Luminex technology described herein and in U.S. Pat. Nos. 6,599,331, 6,592,822, and 6,268,222), or quantum dot technology (for example, as described in U.S. Pat. No. 6,306,610).

In the bead-type immunoassays described in the examples below, the Luminex LabMAP system is utilized. The LabMAP system incorporates polystyrene microspheres that are dyed internally with two spectrally distinct fluorochromes. Using precise ratios of these fluorochromes, an array is created consisting of 100 different microsphere sets with specific spectral addresses. Each microsphere set can possess a different reactant on its surface. Because microsphere sets can be distinguished by their spectral addresses, they can be combined, allowing up to 100 different analytes to be measured simultaneously in a single reaction vessel. A third fluorochrome coupled to a reporter molecule quantifies the biomolecular interaction that has occurred at the microsphere surface. Microspheres are interrogated individually in a rapidly flowing fluid stream as they pass by two separate lasers in the Luminex analyzer. High-speed digital signal processing classifies the microsphere based on its spectral address and quantifies the reaction on the surface in a few seconds per sample.

For the assays described herein, the bead-type immunoassays are preferable for a number of reasons. As compared to ELISAs, costs and throughput are far superior. As compared to typical planar antibody microarray technology (for example, in the nature of the BD Clontech Antibody arrays, commercially available form BD Biosciences Clontech of Palo Alto, Calif.), the beads are superior for quantitation purposes because the bead technology does not require pre-processing or titering of the plasma or serum sample, with its inherent difficulties in reproducibility, cost and technician time. For this reason, although other immunoassays, such as, without limitation, ELISA, RIA, and antibody microarray technologies, are capable of use in the context of the present invention, they are not preferred.

In a preferred embodiment of the invention, the evaluation function comprises a medical team reviewing the results of the testing function. The medical team preferably comprises two or more physicians or other medical practitioners, such as registered nurses. The medical team analyzes the results of a given consumer's biomarker panel assay in order to determine which, if any, of the tested biomarkers are present at concentrations outside the normal range. For those biomarkers that are present outside the normal range, the medical team reviews and discusses the latest information from the medical and research fields, in preparation for briefing the consumer about the implications of the test results.

In a preferred embodiment, the reporting function comprises generating a report of a consumer's individual test results, providing that report to the consumer, and consulting with the consumer regarding the implications of the test results. The report typically highlights, typically via color coded flags, those biomarkers that in a particular consumer's biosample were present at levels outside the normal range. Biomarkers present at levels far outside the normal range are flagged in the color red, designated as the “alert” category, and information about those biomarkers is presented in a prominent section typically near the beginning of the report. Biomarkers present at levels slightly outside, but not far outside, the normal range are flagged in the color yellow, designated as the “caution” category, and information about those biomarkers is presented in a prominent section typically near the beginning of the report. Biomarkers present within the normal range are set to the color green, designated as the “low risk” category, and information about these biomarkers is presented in a “Glossary” section typically near the end of the report. Regarding all of the biomarkers tested as part of the HAS, the information presented in the report includes up-to-date knowledge from the clinical medical and scientific research communities regarding associations between the biomarkers and various medical conditions. The consumer may then use this information to follow up with their personal physician and elect to pursue any prudent counseling, monitoring, preventive lifestyle modifications, or medical treatments.

In another embodiment, a percentage change between levels of a biomarker is compared to RCV for the biomarker. If the percentage change in the level of the biomarker is equal to or higher than the reference change value, then a positive diagnosis of the condition in the patient is ascertained. If the percentage change in level of biomarker is lower than the reference change value, then a negative diagnosis of the condition in the patient is ascertained. These diagnoses can be indicated on the report.

In a preferred embodiment, the reporting function further comprises a consultation, typically over the telephone, with physicians from the HAS Provider. This consultation may, at the consumer's option, be conducted so as to allow the consumer's personal physician to participate. Via the consultation, the HAS Provider physicians will advise the consumer regarding the consumer's overall wellness picture as evidenced by the results of the testing function. For any biomarkers in the “caution” or “alert” categories (i.e., those biomarkers that the testing function revealed were present in the consumer's biosample in concentrations outside the normal range, as defined by the HAS Provider to include, for example, concentrations more than one standard deviation from the range of concentrations observed in a normal population), the HAS Provider physicians typically explain the relevance of the altered biomarker level, including the potential that it signifies the presence or risk for certain medical conditions and how the consumer may proceed to use that information in following up with his personal physician.

Preferred embodiments provide a Health Assessment Service (HAS) comprising:

(i) a marketing function that brings to the attention of potential consumers an ability to purchase an individualized health assessment service;

(ii) a testing function that obtains one or more test samples taken from a consumer who has elected to purchase the service, which one or more samples are subjected to one or more test panels, each of said one or more test panels comprising qualitative and/or quantitative tests for the presence or absence of a plurality of biomarkers in said one or more samples;

(iii) an evaluation function that reviews results from said tests and optionally generates one or more reports; and

(iv) a reporting function that communicates one or more reports to the consumer in a manner that brings to the consumer's attention test results that might inform of disease, medical condition, potential health risks and/or problems, if any.

In another embodiment of the HAS, the test in (ii) is quantitative and comprises

(a) measuring a level of a biomarker in the test sample from the consumer at a first time;

(b) measuring a level of the biomarker in the test sample from the consumer at a second time, wherein the biomarker in (b) are the same as the biomarker in (a);

(c) calculating a percentage change between the level of the biomarker in (a) and (b); and

(d) comparing percentage change in (c) to a reference change value; wherein
(i) percentage change in (c) that is equal to or higher than the reference change value indicates a possibility of a presence of the condition in the consumer; or
(ii) percentage change in (c) that is lower than the reference change value indicates a decreased possibility of a presence of the condition in the consumer.

In another embodiment of the HAS, the reference change value is obtained by

(a) obtaining levels of the biomarker from a population of at least 20 healthy individuals and at least five times from each individual;

(b) using the levels obtained from (a) to determine mean within-subject biological variation (CV_{W});

(c) determining an analytic precision (CV_{A});

(d) determining a bidirectional Z score for desired level of confidence; and

(e) calculating a reference change value (RCV) for the population, wherein RCV=2^{1/2}Z(CV_{A}²+CV_{W}²)^{1/2}, wherein Z is standard deviate appropriate for chosen probability, CV_{A} is analytic precision, CV_{W} is mean within-subject biological variation.

In another embodiment of the HAS, test results are compared against putative reference ranges which are attributed to “normal” ranges.

In another embodiment of the HAS, the reference range for biomarkers of diseases of common prevalence is set at two standard deviations and the reference range for biomarkers of diseases of uncommon prevalence is set at four or more standard deviations.

In another embodiment of the HAS, the one or more test panels include test panels for autoimmune disorder, cancer, cardiovascular disease, cell signaling, diabetes, endocrine, hematology, hormonal imbalance, immune/inflammation, infectious disease, metabolic disorder, nutritional, organ systems, and osteoarthritis.

Preferred embodiment provide a method of providing a Health Assessment Service (HAS) comprising:

(i) soliciting one or more consumers who might be interested in purchasing an individualized health assessment service;

(ii) obtaining one or more test samples taken from a consumer who has elected to purchase the service;

(iii) subjecting the one or more samples to one or more test panels, each of said one or more test panels comprising qualitative and/or quantitative tests for the presence or absence of a plurality of biomarkers in said one or more samples;

(iv) reviewing results from said tests and optionally generating one or more reports; and

(v) communicating one or more reports to the consumer in a manner that brings to the consumer's attention test results that might inform of disease, medical condition, potential health risks and/or problems, if any.

In another embodiment of the method, the one or more consumers exhibit little or no symptoms of disease, medical condition, potential health risks and/or problems.

In another embodiment of the method, the test in (iii) comprises

(a) measuring a level of a biomarker in the test sample from the consumer at a first time;

(b) measuring a level of the biomarker in the test sample from the consumer at a second time, wherein the biomarker in (b) are the same as the biomarker in (a);

(c) calculating a percentage change between the level of the biomarker in (a) and (b); and

(d) comparing the percentage change in (c) to a reference change value; wherein
(i) percentage change in (c) that is equal to or higher than the reference change value indicates a possibility of a presence of the condition in the patient; or
(ii) percentage change in (c) that is lower than the reference change value indicates a decreased possibility of a presence of the condition in the patient.

In another embodiment of the method, the reference change value is obtained by

(a) obtaining levels of the biomarker from a population of at least 20 healthy individuals and at least five times from each individual;

(b) using the levels obtained from (a) to determine mean within-subject biological variation (CV_{W});

(c) determining an analytic precision (CV_{A});

(d) determining a bidirectional Z score for desired level of confidence; and

(e) calculating a reference change value (RCV) for the population, wherein RCV=2^{1/2}Z(CV_{A}²+CV_{W}²)^{1/2}, wherein Z is standard deviate appropriate for chosen probability, CV_{A} is analytic precision, CV_{W} is mean within-subject biological variation.

In another embodiment of the method, test results are compared against putative reference ranges.

In another embodiment of the method, the putative reference ranges are attributed to “normal” ranges.

In another embodiment of the method, the putative reference ranges are established or refined over time.

In another embodiment of the method, a putative reference range is adjusted based on the prevalence of a particular disease or condition in a general population.

In another embodiment of the method, the reference range is adjusted such that the percentage of results in the “abnormal” range inversely correlates with the prevalence of a particular disease or condition in the general population.

In another embodiment of the method, the reference range for biomarkers of diseases of common prevalence is set at two standard deviations and the reference range for biomarkers of diseases of uncommon prevalence is set at four standard deviations.

In another embodiment of the method, the reference range for CA 19-9 as a biomarker for pancreatic cancer is set at four or more standard deviations.

In another embodiment of the method, a putative reference range for a particular biomarker is adjusted to increase specificity at the expense of sensitivity.

Preferred embodiments provide a method of diagnosing a condition selected from the group consisting of autoimmune disorder, cancer, cardiovascular disease, disease and repair associated with cell signaling, diabetes, endocrine condition, hematological abnormality, hormonal imbalance, immune reaction/inflammation, infectious disease, metabolic disorder, malnutrition, impaired organ function, and osteoarthritis in a patient comprising

(a) measuring levels of biomarkers in a test panel comprising one or more of biomarkers in a first sample from a patient at a first time,
wherein the biomarkers are associated with the condition;

(b) measuring levels of biomarkers in a second sample from the patient at a second time, wherein the biomarkers in (b) are the same as the biomarkers in (a);

(c) calculating a percentage change between the levels of biomarkers in (a) and (b); and

(d) comparing the percentage change in (c) to a reference change value; wherein
(i) percentage change in (c) that is equal to or higher than the reference change value indicates a possibility of a presence of the condition in the patient; or
(ii) percentage change in (c) that is lower than the reference change value indicates a decreased possibility of a presence of the condition in the patient.

In another embodiment of the method, the calculation of the range of reference change values for a biomarker comprises

(a) obtaining levels of the biomarker from a population of at least 20 healthy individuals and at least five times from each individual;

(b) using the levels obtained from (a) to determine mean within-subject biological variation (CV_{W});

(c) determining an analytic precision (CV_{A});

(d) determining a bidirectional Z score for desired level of confidence;

(e) calculating a reference change value (RCV) for the population, wherein RCV=2^{1/2}Z(CV_{A}²+CV_{W}²)^{1/2}, wherein Z is standard deviate appropriate for chosen probability, CV_{A} is analytic precision, CV_{W} is mean within-subject biological variation.

The biomarkers listed below in groupings are not limited to the listed conditions. Also, a biomarker listed below in a particular grouping is not limited to the recited condition.

In another embodiment of the method, the condition is autoimmune disease and the biomarker is selected from the group consisting of anti-nuclear antibody, C-reactive protein, double-stranded DNA antibody, ferritin, haptoglobulin, rheumatoid factor, beta-2-glycoprotein, centromere protein B antibody, collagen type 6 antibody, complement factor C1Q antibody, histone antibody, histone H1 antibody, histone H2A antibody, histone H2B antibody, histone H3 antibody, histone H4 antibody, JO-1 antibody, myeloperoxidase antibody, PM-1 antibody, proliferating cell nuclear antigen antibody, proteinase 3 antibody, ribosomal nuclear protein antibody, ribosomal nuclear protein A antibody, ribosomal nuclear protein C antibody, ribosomal P antibody, scleroderma 70 antibody, smith antibody, SSA antibody, and SSB antibody.

In another embodiment of the method, the condition is cancer and the biomarker is selected from the group consisting of basophil count, basophil percentage, beta-2 microglobulin, cancer antigen 125, carcinoembryonic antigen, dihydroepiandrosterone sulfate, eosinophil count, eosinophil percentage, erythropoietin, follicle stimulating hormone, globulin, growth hormone, haptoglobin, Helicobacter pylori IgG antibody, hematocrit, hemoglobin, hepatitis C antibody, human chorionic gonadotropin, immunoglobulin A, immunoglobulin M, insulin, lactate dehydrogenase, luteinizing hormone, lymphocyte count, lymphocyte percentage, monocyte count, monocyte percentage, neutrophil count, neutrophil percentage, platelet count, prolactin, prostate-specific antigen-free, prostate specific antigen-total, testosterone, total protein, white blood cell count, alpha fetoprotein, calcitonin, cancer antigen 15-3, cancer antigen 19-9, endothelin 1, Factor VII, gastrin, glutathione S-transferase, human papilloma virus antibody, and prostatic acid phosphatase.

In another embodiment of the method, the condition is cardiovascular disease and the biomarker is selected from the group consisting of C-reactive protein, cholesterol, creatine kinase MB, creatine kinase-total, ferritin, fibrinogen, haptoglobin, high-density lipoprotein, homocysteine, low-density lipoprotein, low-density lipoprotein/high-density lipoprotein ratio, triglycerides, von Willebrand factor, apolipoprotein A1, B-type natriuretic peptide, endothelin 1, lipoprotein (a), myeloperoxidase antibody, myoglobin, plasminogen activator inhibitor type 1, proliferating cell nuclear antigen antibody, proteinase 3 antibody, apolipoprotein CIII, apolipoprotein H, fatty acid binding protein, fibroblast growth factor-basic form, heat shock cognate protein 70 antibody, heat shock protein 32 antibody, heat shock protein 66 antibody, heat shock protein 71 antibody, heat shock protein 90 alpha antibody, heat shock protein 90 beta antibody, and pregnancy-associated plasma protein A.

In another embodiment of the method, the condition is disease and repair associated with cell signaling and the biomarker is selected from the group consisting of brain-derived neurotrophic factor, eotaxin, epidermal growth factor, fibroblast growth factor-basic form, granulocyte macrophage colony stimulating factor, insulin-like growth factor binding protein 3, insulin-like growth factor 1, intercellular adhesion molecule 1, interleukin-1 alpha, interleukin-1 beta, interleukin-2, interleukin-3, interleukin-4, interleukin-5, interleukin-6, interleukin-7, interleukin-8, interleukin-10, interleukin-12 p40, interleukin-12 p70, interleukin-13, interleukin-15, interleukin-16, interleukin-18, lymphotactin, macrophage-derived chemokine, macrophage inflammatory protein 1 alpha, macrophage inflammatory protein 1-beta, matrix metalloproteinase 2, matrix metalloprotein-3, matrix, metalloprotein-9, monocyte chemotactic protein-1, regulated upon activation, normal T-cell expressed and secreted, stem cell factor, tissue inhibitor of metalloproteinase 1, tumor necrosis factor alpha, tumor necrosis factor beta, tumor necrosis factor receptor 2, vascular cell adhesion molecule 1, and vascular endothelial growth factor.

In another embodiment of the method, the condition is diabetes and the biomarker is selected from the group consisting of glucose, insulin, insulin antibody, C-peptide, hemoglobin A1c, leptin, pancreatic islet cell antibody, adiponectin, insulin-like growth factor binding protein 3, and insulin-like growth factor-1.

In another embodiment of the method, the condition is an endocrine condition and the biomarker is selected from the group consisting of cortisol, follicle stimulating hormone, growth hormone, luteinizing hormone, prolactin, thyroid stimulating hormone, calcium, parathyroid hormone, phosphorus, thyroglobulin antibody, thyroid microsomal antibody, thyroid stimulating hormone, thyroxine, thyroxine binding globulin, triiodothyronine, calcitonin, thyroglobulin antigen, thyroxine antibody, triiodothyronine antibody, dihydroepiandrosterone sulfate, estradiol, follicle stimulating hormone, luteinizing hormone, progesterone, prolactin, testosterone, androstenedione, estriol, unconjugated, and sex hormone-binding globulin.

In another embodiment of the method, the condition is a hematological abnormality and the biomarker is selected from the group consisting of bilirubin-total, eosinophil count, eosinophil percentage, erythropoietin, ferritin, fibrinogen, hematocrit, hemoglobin, iron binding capacity-total, iron-serum, lactate dehydrogenase, lymphocyte count, lymphocyte percentage, mean corpuscular hemoglobin, mean corpuscular hemoglobin concentration, mean corpuscular volume, monocyte count, monocyte percentage, platelet count, red blood cell count, red cell distribution width, vitamin B-12, white blood cell count, basophil count, basophile percentage, Factor VII, haptoglobin, thromobopoietin, tissue factor, and von Willebrand factor.

In another embodiment of the method, the condition is an immune reaction or an inflammatory response and the biomarker is selected from the group consisting of aspartate aminotransferase antigen, C-reactive protein, haptoglobin, immunoglobulin A, immunoglobulin E, immunoglobulin M, von Willebrand factor, Factor VII, alpha-2-macroglobulin, complement-3, epithelial neutrophil activating peptide 78, heat shock cognate protein 70 antibody, heat shock protein 32 antibody, heat shock protein 65 antibody, heat shock protein 71 antibody, heat shock protein 90 alpha antibody, heat shock protein 90 beta antibody, and serum amyloid P.

In another embodiment of the method, the condition is an infectious disease and the biomarker is selected from the group consisting of Helicobacter pylori IgG antibody, Mycoplasma pneumoniae antibody, Streptolysin 0 antibody, Bordetella pertussis antibody, Campylobacter jejuni antibody, Chlamydia pneumoniae antibody, Chlamydia trachomatis antibody, Diphtheria toxin antibody, Leishmania donovani antibody, Lyme disease antibody, Mycobacteria tuberculosis antibody, Tetanus antibody, Toxoplasma gondi antibody, Trypanosoma cruzi antibody, Cytomegalovirus antibody, Epstein-Barr virus early antigen antibody, Hepatitis A antibody, Hepatitis B core antibody, Hepatitis B e antibody, Hepatitis B surface antibody, Hepatitis B surface antigen, Hepatitis C antibody, Hepatitis D antibody, Hepatitis E orf 2.3 kD antibody, Hepatitis orf 2.6 kD antibody, Hepatitis orf 3.3 kD antibody, Influenza A H3N2 antibody, Rubella antibody, Rubeola antibody, Varicella zoster IgG antibody, Varicella zoster IgM antibody, Adenovirus antibody, Herpes simplex virus type 1 glycoprotein D antibody, Herpes simplex virus type 2 glycoprotein G antibody, Herpes simplex virus types 1 and 2 antibodies, human papilloma virus antibody, human T-cell lymphotropic virus types 1 and 2 antibodies, influenza A antibody, influenza B antibody, mumps antibody, parainfluenza type 1 antibody, parainfluenza type 2 antibody, parainfluenza type 3 antibody, polio antibody, respiratory syncytial virus antibody, Epstein-Barr nuclear antigen antibody, and Epstein-Barr viral capside antigen antibody.

In another embodiment of the method, the condition is malnutrition and the biomarker is selected from the group consisting of albumin, albumin/globulin ratio, amylase, calcium, carbon dioxide, chloride, cholesterol, ferritin, folic acid, globulin, glucose, hematocrit, hemoglobin, iron binding capacity-total, iron binding capacity-unsaturated, iron-percent saturated, iron-serum, magnesium, mean corpuscular hemoglobin, mean corpuscular hemoglobin concentration, phosphorus, potassium, sodium, total protein, triglycerides, uric acid, and vitamin B-12.

In another embodiment of the method, the condition is impaired organ function and the biomarker is selected from the group consisting of Helicobacter pylori IgG antibody, Campylobacter jejuni antibody, anti-Saccharomyces cerevisiae antibody, gastin, tissue transglutaminase antibody, blood urea nitrogen, blood urea nitrogen/creatinine ratio, carbon dioxide, chloride, creatinine, potassium, sodium, uric acid, beta-2-microglobulin, alanine aminotransferase, alkaline phosphatase, aspartate aminotransferase, bilirubin-total, ferritin, fibrinogen, gamma glutamyl transferase, haptoglobin, hepatitis A antibody, hepatitis B core antibody, hepatitis B e antibody, hepatitis B surface antibody, hepatitis B surface antigen, hepatitis C antibody, hepatitis D antibody, hepatitis E orf 2.3 antibody, hepatitis E orf 2.6 antibody, hepatitis E orf 3.3 antibody, iron binding capacity-total, lactate dehydrogenase, alpha-1-antitrypsin, cytochrome P-450 antibody, glutathione S-transferase, and mitochondrial antibody.

In another embodiment of the method, the condition is osteoarthritis and the biomarker is selected from the group consisting of C-reactive protein, ferritin, haptoglobin, rheumatoid factor, von Willebrand factor, anti-nuclear antibody, collagen type 1 antibody, collagen type 2 antibody, collagen type 4 antibody, collagen type 6 antibody, heat shock cognate protein 70 antibody, heat shock protein 32 antibody, heat shock protein 65 antibody, heat shock protein 71 antibody, heat shock protein 90 alpha antibody, and heat shock protein 90 beta antibody.

In another embodiment of the method, at least 2 biomarkers are measured.

In another embodiment of the method, at least 5 biomarkers are measured.

In another embodiment of the method, at least 10 biomarkers are measured.

In another embodiment of the method, the sample is serum, blood, urine, saliva, a cell, or a portion of tissue.

### EXAMPLES

### Example 1

### Sample Collection and Assay

Collection and storage of blood specimens: 5 mL of peripheral blood was drawn from subjects using standardized phlebotomy procedures. Blood samples were collected without anticoagulant into one 5 mL red top vacutainer, sera were separated by centrifugation, and all specimens were immediately frozen and stored in the dedicated −80° C. freezer. All blood samples were logged on the HAS Provider's laboratory computer to track information such as storage date, freeze/thaw cycles and distribution.

Spotting and processing of blood specimens: Filter papers containing blood spots were removed from storage if necessary and examined to determine if the blood stain covers an area sufficient to punch with a hole puncher three 6.2 mm diameter holes entirely containing blood stain. Three 6.2 mm diameter holes were punched that were completely stained with blood. The three punched out holes were then placed into a separation device, such that the punched out holes were situated flat against the bottom of the separation device. The separation device in turn was placed in a 1.5 mL microcentrifuge tube. One hundred thirty microliters PBS-4% BSA elution buffer was added to each separation device, such that the elution buffer completely covered the blood spots. Care was exercised not to force liquid past the media in the separation device. Spots were allowed to soak for at least 5 minutes at room temperature. The tubes were then gently vortexed to mix the elution buffer with the spots, while avoiding forceful shaking that would have resulted in allowing liquid to pass through separation device. The tubes were covered using plate cover film but were not capped, as capping creates pressure that could have forced the liquid through the separation device. The microcentrifuge tubes containing the separation devices, elution buffer, and punched out blood spot holes were placed on a microcentrifuge and spun for a minimum 12 hours, at 2-8° C. Samples were removed from the centrifuge and the cover film was carefully removed from the samples. The tubes were capped and placed back in the centrifuge and spun at 14,000 rpm, for a minimum of 1 minute, at 2-8° C. The devices were removed from the centrifuge and each microcentrifuge tube was inspected to confirm complete elution, such that liquid was at the bottom of the microcentrifuge tube and the filter paper appeared almost dry and with no traces eluent. The separation devices were removed from the 1.5 mL tubes and discarded. The microcentrifuge tubes containing eluent were recapped and stored at 2-8° C. until further processing.

Development of Luminex assays: Luminex assays were developed to efficiently and accurately test the majority of the biomarkers described in Table 1. Luminex technology is described in the art and incorporated herein by reference.

Serum concentrations of biomarkers. Circulating concentrations of different serum biomarkers were evaluated in multiplexed assays using LabMap technology in blood of individuals that elected to utilize the HAS.

### Example 2

### Calculation of RCV

Illustrated below is a sample calculation of an RCV.

Patient A's PSA level is measured as a baseline or reference value. A number is reported, for example, 1.1 ng/mL. The value 1.1 ng/mL can be reported along with a reference range (taken from the laboratory's experience of the population as a whole), which says that a value under 4.0 ng/mL indicates negative diagnosis for prostate cancer. Thus, the conclusion is that Patient A is normal.

Patient A goes back with a follow up visit a year later and gets measured for PSA again. The laboratory can conduct a procedure for drawing sample, handling, instrument, etc. in the same way as the year before to minimize pre-analytical variation and analytical variation. Patient A is interviewed to establish that change in patient variation (e.g., sick when sample is taken, suffered a trauma) is not a factor.

Results for the follow up visit come back with PSA level as 1.8 ng/mL. This result is a 64% change from the year before. Thus, the percentage change in PSA level is 64%.

Using the equation for RCV: RCV=2^{1/2}Z(CV_{A}²+CV_{W}²)^{1/2}, wherein 2^{1/2} is 1.414, Z is 1.96 with 95% confidence or 2.58 with 99% confidence, CV_{A}² and CV_{W}² can be obtained from analysis of variance. In calculation of RCV for PSA, there is a 95% confidence level that a 57% or higher is a significant change from last year and there is a 99% confidence level that a 75% change or higher is highly significant.

Thus, the 64% change in PSA levels in Patient A is higher than RCV at 95% confidence level. Patient A is advised that there has been a significant change in his PSA values from the year before and patient gets more diagnostics, biopsy, or evaluations. Traditional method would have concluded that Patient A is fine (i.e., under the reference range of 4.0 ng/mL), whereas RCV calculation and comparison indicates that more investigation is recommended.

While the above detailed description has shown, described and identified several novel features of the invention as applied to a preferred embodiment, it will be understood that various omissions, substitutions and changes in the form and details of the described embodiments may be made by those skilled in the art without departing from the spirit of the invention. Accordingly, the scope of the invention should not be limited to the foregoing discussion, but should be defined by the appended claims.

What is claimed is:

1. A Health Assessment Service (HAS) comprising:
(i) a marketing function that brings to the attention of potential consumers an ability to purchase an individualized health assessment service;
(ii) a testing function that obtains one or more test samples taken from a consumer who has elected to purchase the service, which one or more samples are subjected to one or more test panels, each of said one or more test panels comprising qualitative and/or quantitative tests for the presence or absence of a plurality of biomarkers in said one or more samples;
(iii) an evaluation function that reviews results from said tests and optionally generates one or more reports; and
(iv) a reporting function that communicates one or more reports to the consumer in a manner that brings to the consumer's attention test results that might inform of disease, medical condition, potential health risks and/or problems, if any.

2. The HAS of claim 1 in which the one or more test panels include test panels for autoimmune disorder, cancer, cardiovascular disease, cell signaling, diabetes, endocrine, hematology, hormonal imbalance, immune/inflammation, infectious disease, metabolic disorder, nutritional, organ systems, and osteoarthritis.

3. A method of providing a Health Assessment Service (HAS) comprising:
(i) soliciting one or more consumers who might be interested in purchasing an individualized health assessment service;
(ii) obtaining one or more test samples taken from a consumer who has elected to purchase the service;
(iii) subjecting the one or more samples to one or more test panels, each of said one or more test panels comprising qualitative and/or quantitative tests for the presence or absence of a plurality of biomarkers in said one or more samples;
(iv) reviewing results from said tests and optionally generating one or more reports; and
(v) communicating one or more reports to the consumer in a manner that brings to the consumer's attention test results that might inform of disease, medical condition, potential health risks and/or problems, if any.

4. The method of claim 3 in which the one or more consumers exhibit little or no symptoms of disease, medical condition, potential health risks and/or problems.

5. The method of claim 3, wherein the test in (iii) comprises
(a) measuring a level of a biomarker in the test sample from the consumer at a first time;
(b) measuring a level of the biomarker in the test sample from the consumer at a second time, wherein the biomarker in (b) are the same as the biomarker in (a);
(c) calculating a percentage change between the level of the biomarker in (a) and (b); and
(d) comparing the percentage change in (c) to a reference change value; wherein
(i) percentage change in (c) that is equal to or higher than the reference change value indicates a possibility of a presence of the condition in the patient; or
(ii) percentage change in (c) that is lower than the reference change value indicates a decreased possibility of a presence of the condition in the patient.

6. The HAS of claim 5, wherein the reference change value is obtained by
(a) obtaining levels of the biomarker from a population of at least 20 healthy individuals and at least five times from each individual;
(b) using the levels obtained from (a) to determine mean within-subject biological variation (CV_{W});
(c) determining an analytic precision (CV_{A});
(d) determining a bidirectional Z score for desired level of confidence; and
(e) calculating a reference change value (RCV) for the population, wherein RCV=2^{1/2}Z(CV_{A}²+CV_{W}²)^{1/2}, wherein Z is standard deviate appropriate for chosen probability, CV_{A} is analytic precision, CV_{W} is mean within-subject biological variation.

7. The method of claim 3 in which test results are compared against putative reference ranges.

8. The method of claim 7 in which the putative reference ranges are attributed to “normal” ranges.

9. The method of claim 7 in which the putative reference ranges are established or refined over time.

10. The method of claim 7 in which a putative reference range is adjusted based on the prevalence of a particular disease or condition in a general population.

11. The method of claim 10 in which the reference range is adjusted such that the percentage of results in the “abnormal” range inversely correlates with the prevalence of a particular disease or condition in the general population.

12. The method of claim 11 in which the reference range for biomarkers of diseases of common prevalence is set at two standard deviations and the reference range for biomarkers of diseases of uncommon prevalence is set at four standard deviations.

13. The method of claim 12 in which the reference range for CA 19-9 as a biomarker for pancreatic cancer is set at four or more standard deviations.

14. The method of claim 7 in which a putative reference range for a particular biomarker is adjusted to increase specificity at the expense of sensitivity.

15. A method of diagnosing a condition selected from the group consisting of autoimmune disorder, cancer, cardiovascular disease, disease and repair associated with cell signaling, diabetes, endocrine condition, hematological abnormality, hormonal imbalance, immune reaction/inflammation, infectious disease, metabolic disorder, malnutrition, impaired organ function, and osteoarthritis in a patient comprising
(a) measuring levels of biomarkers in a test panel comprising one or more of biomarkers in a first sample from a patient at a first time,
wherein the biomarkers are associated with the condition;
(b) measuring levels of biomarkers in a second sample from the patient at a second time, wherein the biomarkers in (b) are the same as the biomarkers in (a);
(c) calculating a percentage change between the levels of biomarkers in (a) and (b); and
(d) comparing the percentage change in (c) to a reference change value; wherein
(i) percentage change in (c) that is equal to or higher than the reference change value indicates a possibility of a presence of the condition in the patient; or
(ii) percentage change in (c) that is lower than the reference change value indicates a decreased possibility of a presence of the condition in the patient.

16. The method of claim 15, wherein the calculation of the range of reference change values for a biomarker comprises
(a) obtaining levels of the biomarker from a population of at least 20 healthy individuals and at least five times from each individual;
(b) using the levels obtained from (a) to determine mean within-subject biological variation (CV_{W});
(c) determining an analytic precision (CV_{A});
(d) determining a bidirectional Z score for desired level of confidence;
(e) calculating a reference change value (RCV) for the population, wherein RCV=2^{1/2}Z(CV_{A}²+CV_{W}²)^{1/2}, wherein Z is standard deviate appropriate for chosen probability, CV_{A} is analytic precision, CV_{W} is mean within-subject biological variation.

17. The method of claim 15, wherein at least 2 biomarkers are measured.

18. The method of claim 15, wherein at least 5 biomarkers are measured.

19. The method of claim 15, wherein at least 10 biomarkers are measured.

20. The method of claim 15, wherein the sample is serum, blood, urine, saliva, a cell, or a portion of tissue.
